(19) United States
(12) Patent Application Publication
(10) Pub. No.: US 2017/0368298 A1
(43) Pub. Date: Dec. 28, 2017

(54) FACE PLUNGE DEVICE AND METHODS FOR TRIGGERING THE CENTRAL NERVOUS SYSTEM AND PROMOTING HUMAN HEALTH AND BEAUTY BENEFITS USING SAME

(71) Applicant: Dinka Bojanova, Kilauea, HI (US)

(72) Inventor: Dinka Bojanova, Kilauea, HI (US)

(21) Appl. No.: 15/636,312

(22) Filed: Jun. 28, 2017

Related U.S. Application Data
(60) Provisional application No. 62/355,720, filed on Jun. 28, 2016.

(51) Int. Cl. A61M 21/02; A61H 35/00; A45D 44/00; A61F 7/00; A61N 5/06; A61M 21/00

(52) U.S. Cl. CPC A61M 21/02; A61F 7/007; A61N 5/0622; A61H 35/008; A45D 44/00; A61F 2007/0003; A61H 2201/0221; A61M 2021/0066; A61N 2005/0663; A61H 2201/0214

(57) ABSTRACT

The present disclosure describes a manual or automated face plunge comprising a water reservoir, and optionally a chilling apparatus comprising one or more thermoelectric coolers to chill the water, and a controller to electronically control the chilling apparatus. The disclosure further describes methods for chilling the face, triggering the central nervous system, promoting human health, promoting human beauty, and promoting aesthetic benefits to a human in need thereof, comprising immersing the face of the person in the face plunge in accordance to various immersion regimens. In various aspects, the face plunge comprises ice-cold water for the facial immersions.

### CROSS-REFERENCE TO RELATED APPLICATIONS

This application claims priority to U.S. Provisional Patent Application Ser. No. 62/355,720, filed Jun. 28, 2016, the disclosure of which is incorporated herein by reference it its entirety.

### FIELD OF THE INVENTION

The present disclosure relates generally to health and beauty appliances and methods of use to promote human health and beauty, and in particular to a face plunge device optimized for the ice-cold water immersion of the human face, and methods for chilling the face, triggering the central nervous system, and promoting human health and beauty benefits using said face plunge.

### BACKGROUND OF THE INVENTION

Ice water treatment has been known for generations to soothe aches and pains and reduce inflammation in humans. More recently, cold water immersion has been shown to affect physiological changes in the human body possibly leading to overall benefits such as reducing frequency of the common cold, reducing insomnia, chronic fatigue syndrome, heart failure and cancer, and increasing metabolism, immunity, relieve depression, boost mood and energy. Without question, an icy cold water plunge is invigorating.

Physiologically in humans, cold water immersion has been shown to increase interleukin-6 (IL-6) levels, induce leukocytosis, and increase natural killer (NK) cell activity, (see, e.g. K. M. Brenner, et al., J. App. Phys., 87(2), 699-710, 1999). These and perhaps other biochemical and cellular level changes may be the underlying drivers for the observed health benefits of cold water immersion.

However, notwithstanding privileged access for some individuals to a frozen pond, cold water immersion requires a cold water spa. Such a spa can be cost prohibitive to the average consumer, too bulky for practical household installation in most homes, and overly time consuming to fill, bring to temperature, and use.

Therefore, there is a continuing need for more practical devices, such as home appliances, which can bring about the same or similar physiological changes in the human body as does the full body cold water immersion. There is also a continuing need to discover precise regimens for chilling the body, or even just portions of the body, to invigorate the body and promote health benefits in a human.

### SUMMARY OF THE INVENTION

Suspecting that cold water immersion of just the face of a human, which is close in proximity to the brain and central nervous system (CNS), could promote similar health benefits as the full body cold water immersion, a practical face plunge appliance has been developed, the details of which are disclosed herein. For example, one study showed that total immersion of the body in water had the same effect as facial immersion. The high receptor density in the human face may explain this effect. The most important parts of the face to submerge are the areas around the forehead, nose, and eyes because of the high receptor density in these areas. This is likely due to the ophthalmic division of the trigeminal nerve, which is very sensitive to cold water, (see Parfrey, P. and Sheehan, J. D., “Individual facial areas in the human circulatory response to immersion,” Irish Journal of Medical Science, 1975; 144: 335-342). Furthermore, methods for chilling the human face and promoting human health and beauty benefits are disclosed using this inventive face plunge in accordance to various treatment regimens. The methods disclosed herein comprise immersion procedures easily followed by the person using the face plunge appliance, even in its simplest embodiment as a bowl of chilled water, with the results being a triggering of the CNS, invigoration, stimulation, promotion of general health benefits and promotion of general beauty benefits, such as beautiful skin appearance.

In various embodiments, manual and automatic face plunge devices are disclosed. In certain aspects, a manual face plunge device comprises a bowl, insulated or otherwise, containing chilled water. In other aspects, an automatic face plunge device comprises a water reservoir, e.g. dimensioned to the human face, a chilling apparatus, and a controller for controlling the chilling apparatus and consequently the temperature of the water in the reservoir. The chilling apparatus may comprise at least one thermoelectric cooler, and optionally, any number of heat sinks and/or fans to move heat from the water to the ambient surroundings. The controller may comprise digital electronics, such as a microprocessor and various LED displays. The controller may afford timing options for start-up of the device, and particular temperature ranges and temperature uniformity for the water in the reservoir. For example, the controller may allow the user to set a particular time in the morning at which the water in the device will be chilled and ready for use. The face plunge may be self-sanitizing by built-in uv irradiation, and may feature many other attributes as disclosed herein.

In various aspects of the present disclosure, a face plunge device for chilling the face of a human is disclosed. The face plunge device comprises: a primary reservoir having an interior surface and an exterior surface, said primary reservoir containing water; a chilling apparatus configured to chill the water; and a controller configured to electronically control the chilling apparatus, wherein immersion of the face in the water for a period of time of from about 1 second to about 10 seconds lowers the temperature of the skin of the face by at least 1° F. In some aspects, a primary reservoir may comprise a capacity of from about 0.5 liters to about 10 liters. The controller may be used to thermostatically maintain a temperature of the water in the primary reservoir at from about 32° F. to about 60° F.

In some examples, a chilling apparatus may comprise at least one thermoelectric cooler (TEC) disposed in close proximity to, or in contact with, the exterior surface of the primary reservoir.

In certain aspects, a face plunge device in accordance to the present disclosure may further comprise a secondary reservoir in fluid communication with the primary reservoir. The chilling apparatus may be configured to chill water present in the secondary reservoir and to circulate the water between the primary and secondary reservoirs via a pump. In this way, a single body of water is circulated between primary and secondary reservoirs. The chilling apparatus may further comprise at least one thermoelectric cooler (TEC) disposed in close proximity to, or in contact with, the secondary reservoir.

In other configurations, the primary reservoir comprises a two-layer structure of inner and outer layers, wherein an innermost interior surface of the inner layer defines a volume of the primary reservoir, the outermost exterior surface of the outer layer defines an outer shape of the primary reservoir, and a space between the interior and exterior layers contains a refrigerant gel, some other liquid, air or insulation. In other configurations, the space contains a cooling apparatus to child the water in the primary reservoir.

In certain examples, a face plunge device further comprises a visible light source configured and disposed to project visible light into the primary reservoir, said visible light having wavelengths in the range of from about 450 to about 495 nm or from about 620 to about 750 nm. The visible light is seen by the person using the face plunge device, wherein the visible light simulates the person's pineal gland.

In accordance to other aspects of the present disclosure, a method of non-invasive therapeutic treatment is disclosed. The method of non-invasive therapeutic treatment in a human comprises immersing the face of the human in need thereof in the face plunge device for a period of time of from about 1 to about 10 seconds, or for a period of time sufficient to lower the temperature of the skin of the face by at least 1° F., wherein the temperature of the water into which the face is immersed is at about 32° F. to about 60° F. prior to immersion. In certain aspects, a use regimen comprises repeating the immersion 3 to 5 times with a brief pause between immersions.

In certain aspects, the non-invasive therapeutic treatment comprises at least one of triggering the central nervous system, resetting circadian rhythm, increasing anti-inflammatory cytokine production and/or levels, decreasing inflammatory cytokine production and/or levels, increasing glutathione production and/or levels, increasing antioxidant production and/or levels, flushing out of toxins from skin connective tissue, inhibiting melanogenesis, increasing synthesis of collagen, reducing frequency/duration of colds, reducing pain, reducing swelling, reducing sinus congestion, reducing frequency/severity of acne, reducing eye redness, reducing eye irritation, reducing psoriasis, improving sleep, reducing sleep apnea, reducing severity and duration of symptoms of depression, reducing cancer, reducing blood pressure levels, reducing heart rate, and reducing pulse rate. Anti-inflammatory cytokines may comprise at least one of IL-1 receptor antagonist, IL-4, IL-10, IL-11, IL-13 and TGF-β, and inflammatory cytokines may comprise at least one of IL-1, TNF-α, and IFN-γ. When the face plunge device further comprises a visible light source as mentioned, the device may be used in a method of additional stimulus of the pineal gland of a human in need thereof.

In various embodiments, symptoms of depression that are alleviated by using the face plunge herein comprise at least one of anxiety, apathy, general discontent, guilt, hopelessness, loss of interest, loss of interest or pleasure in activities, mood swings, sadness, early awakening, excessive sleepiness, insomnia, restless sleep, excessive hunger, fatigue, loss of appetite, restlessness, agitation, excessive crying, irritability, social isolation, lack of concentration, slowness in activity, thoughts of suicide, weight gain, weight loss, poor appetite, and repeatedly going over thoughts.

In various embodiments of the present disclosure, a method of beauty treatment in a human is disclosed. The method of beauty treatment in a human comprises immersing the face of the human in need thereof in the face plunge for a period of time of from about 1 to about 10 seconds, or for a period of time sufficient to lower the temperature of the skin of the face by at least 1° F., wherein the temperature of the water into which the face is immersed is at about 32° F. to about 60° F. prior to immersion. In various examples, the immersion is repeated 3 to 5 times with a brief pause between immersions.

The beauty treatment may comprise at least one of reducing skin redness, moisturizing and hydrating skin, improving skin clarity, reducing freckles, reducing age spots, reducing skin dryness, reducing fine lines, reducing pore size, improving skin smoothness, improving skin tone, reducing wrinkles, increasing skin firmness, increasing skin softness, stimulating production of new skin cells, and improving skin suppleness.

In other aspects of the disclosure, a method of invigorating a human is disclosed. The method of invigorating a human comprises immersing the face of the human in need thereof in the face plunge device of claim 1 for a period of time of from about 1 to about 10 seconds, or for a period of time sufficient to lower the temperature of the skin of the face by at least 1° F., wherein the temperature of the water into which the face is immersed is at about 32° F. to about 60° F. prior to immersion. In certain examples, the immersion may be repeated 3 to 5 times with a brief pause between immersions.

In other aspects of the disclosure, a method of achieving instant alertness in a human is disclosed, bringing the wondering human mind into the present moment. The method of instant alertness in a human comprises of immersing the face of the human in need thereof in the face plunge device herein for a period of time sufficient to lower the temperature of the skin of the face by at least 1° F., wherein the temperature of the water into which the face is immersed is at about 32° F. to about 60° F. prior to immersion. In certain examples, the immersion may be repeated 3 to 5 times with a brief pause between immersions.

In other aspects of the disclosure, a method of mending the circadian rhythm in a human is disclosed. The method of mending the circadian rhythm in a human comprises immersing the face of the human in need thereof in the face plunge device herein for a period of time sufficient to lower the temperature of the skin of the face by at least 1° F., wherein the temperature of the water into which the face is immersed is at about 32° F. to about 60° F. prior to immersion. In certain examples, the immersion may be repeated 3 to 5 times with a brief pause between immersions.

In other aspects of the disclosure, a method of increasing energy in a human is disclosed. The method of increasing energy a human comprises immersing the face of the human in need thereof in the face plunge device of claim 1 for a period of time of from about 1 to about 10 seconds, or for a period of time sufficient to lower the temperature of the skin of the face by at least 1° F., wherein the temperature of the water into which the face is immersed is at about 32° F. to about 60° F. prior to immersion. In certain examples, the immersion may be repeated 3 to 5 times with a brief pause between immersions.

### BRIEF DESCRIPTION OF THE DRAWINGS

The accompanying drawings are included to provide further understanding of the disclosure and are incorporated in and constitute a part of this specification, illustrate embodiments of the disclosure, and together with the description serve to explain the principles of the disclosure, wherein:

FIG. 1 illustrates an embodiment of a face plunge in accordance with the present disclosure, with various engineering aspects detailed;

FIG. 2 illustrates an embodiment of a face plunge in accordance with the present disclosure, with various engineering aspects detailed;

FIG. 3 illustrates an embodiment of a face plunge in accordance with the present disclosure;

FIG. 4 illustrates an embodiment of a face plunge in accordance with the present disclosure;

FIG. 5 illustrates an embodiment of a face plunge in accordance with the present disclosure;

FIG. 6 illustrates an embodiment of a face plunge in accordance with the present disclosure;

FIG. 7 illustrates an embodiment of a face plunge in accordance with the present disclosure;

FIG. 8 illustrates an embodiment of a face plunge in accordance with the present disclosure;

FIG. 9 illustrates an embodiment of a face plunge in accordance with the present disclosure;

FIG. 10 illustrates an embodiment of a face plunge in accordance with the present disclosure;

FIG. 11 illustrates an embodiment of a face plunge in accordance with the present disclosure, with various engineering aspects detailed;

FIG. 12 illustrates an embodiment of a face plunge in accordance with the present disclosure, with various engineering aspects detailed; and

FIG. 13 illustrates an embodiment of a primary reservoir in accordance with the present disclosure, the features of which can be interchanged and combined as necessary for a particular face plunge appliance.

### DETAILED DESCRIPTION OF THE INVENTION

The following description is of various exemplary embodiments only, and is not intended to limit the scope, applicability or configuration of the present disclosure in any way. Rather, the following description is intended to provide a convenient illustration for implementing various embodiments including the best mode. As will become apparent, various changes may be made in the function and arrangement of the elements described in these embodiments without departing from principles of the present disclosure.

As described in more detail herein, various embodiments of the present disclosure generally comprise a face plunge device and methods for chilling the face using same. Further methods comprise promotion of health and beauty benefits in a human subject by chilling the face of the subject in the ice-cold water of the face plunge in accordance to prescribed regimens. General health benefits include, but are not limited to, such subjective measures as feeling better, having more energy, or simply feeling more invigorated when using the face plunge of the present disclosure.

In various embodiments, the present disclosure provides methods of therapeutic treatment. In various aspects, methods of non-invasive therapeutic treatment are provided. Methods of non-invasive therapeutic treatment include, but are not limited to, methods to trigger the central nervous system, reset circadian rhythm, increase anti-inflammatory cytokine production and/or levels, decrease inflammatory cytokine production and/or levels, increase glutathione production and/or levels, increase antioxidant production and/or levels, flush out of toxins from skin connective tissue, inhibit melanogenesis, increase synthesis of collagen, reduce frequency/duration of colds, reduce pain, reduce swelling, reduce sinus congestion, reduce frequency/severity of acne, reduce eye redness, reduce eye irritation, reduce psoriasis, improve sleep, reduce sleep apnea, reduce severity and duration of symptoms of depression, reduce cancer, stimulate the pineal gland, reduce blood pressure levels, reduce heart rate, and reduce pulse rate. These methods, and others, utilize the face plunge device as described herein.

The present disclosure also provides methods of beauty treatment. Methods of beauty treatment include, but are not limited to, methods to reduce skin redness, moisturize and hydrate skin, improve skin clarity, reduce freckles, reduce age spots, reduce skin dryness, reduce fine lines, reduce pore size, improve skin smoothness, improve skin tone, reduce wrinkles, increase skin firmness, increase skin softness, stimulating production of new skin cells, and improve skin suppleness. These methods, and others, utilize the face plunge device as described herein.

The present disclosure also provides a finishing protocol for a person at the end of a spa treatment, for example a facial spa treatment.

As used herein, the term “ice-cold face plunge,” or more simply, “face plunge,” refers to a device capable of containing a volume of water at from about 32° F. to about 60° F. in accordance with the present disclosure. The face plunge herein may be categorized as being either “automatic,” meaning the device comprises at least some electronics, or “manual,” meaning the device comprises a bowl for retaining water, and optionally some type of insulation, a lid, and decoration. The differences between the automatic face plunge and the manual face plunge are discussed in more detail herein. It's important to note that the methods for non-invasive therapeutic treatment and methods for beauty treatment may be enabled by any embodiment of the face plunge, regardless if automatic or manual.

The ice-cold face plunge herein may be compact in size, e.g. a “table-top” version sized for use on a counter surface or table, or it may be fitted to a pedestal such that the overall appliance resembles a household pedestal sink and can stand alone at a convenient height without placement on a counter. In other aspects, the appliance may be built into the plumbing and/or electrical system of a home or institution as a permanent or semi-permanent fixture. The appliance disclosed herein may be battery powered, solar powered, or powered by any voltage/current, such as 110 Volts AC and/or 220 Volts AC, (at any amperage/wattage rating). For example, the appliance may be equipped with a power cord terminating in the appropriate electric plug configured for a particular country. The face plunge of the present invention may be found and used in a residential home, group home, hotel room, hotel spa, fitness and health club, beauty school, public school, college or university, beauty salon, nail salon, and the like.

The manual face plunge device may be as simple as a plastic bowl, optionally equipped with a lid. In some embodiments detailed herein, a manual face plunge device may comprise a bowl with various layers, such as to provide insulation so that the water in the bowl remains at a desired temperature for at least the period of time during facial immersions.

As used herein, the term “chilling the face” refers to reducing the temperature of the skin of a human face. In certain examples, chilling the face comprises a reduction in facial skin temperature of 1° F. or more. After immersion of a human face in the face plunge herein, and subsequent removal therefrom, a reduction in skin temperature may be detected, which is the difference between the skin temperature immediately prior to immersion and the skin temperature immediately after immersion. This temperature drop can be measured, for example, by pressing a thermometer against the face or by aiming an infrared temperature sensor at the face, taking readings before and after immersion. An exemplary method of chilling a human face comprises immersing the face in the face plunge further comprising ice-cold water, wherein skin temperature of the face is reduced by at least 1° F.

As used herein, the term “ice-cold” is used in reference to the temperature of the water of the face plunge device, and refers to either the actual temperature of the water, or the sensory perception of that water, namely that it is close to the freezing point of water, 32° F., or at least less than or equal to about 60° F., as measured, or that it is perceived as being quite cold. Thus, “ice-cold” can be a subjective term herein, relating to the sensory perception of how cold the water is in the face plunge device. For example, a person plunging their face into 60° F. or less water may proclaim that the water is “ice-cold.” Herein, the term “ice-cold” water is broadly used to indicate water having a temperature of from about 32° F. to about 60° F., even though the water may not have any perceivable ice.

As used herein, the term “invigorating” refers to any subjective feeling in an individual relating to energy, being more awake, more alert, less depressed, enlightened, euphoric, and so forth. Thus, a human subject may feel “invigorated” after submerging his/her face in the ice-cold face plunge of the present disclosure, if, by doing so, that individual now feels more awake, alert, energetic, happy, enlightened, euphoric, etc. In various aspects, immersion of the face into the ice-cold face plunge creates a total sensory experience for the user. Immersion of the face into the face plunge may replace the feeling an astringent is desired on the skin of the face, such as to give the sensation of cooling and refreshing as would an astringent like alcohol or witch hazel.

As used herein, the term “health benefits” refers broadly to any improvement in human health using the appliance herein, such as those that are macroscopically observable (e.g. reduction in frequency/duration of colds, reduced pain, reduced swelling, reduced sinus congestion, reduction in the frequency and severity of acne, reduced psoriasis, improved sleep, reduced sleep apnea, reduced signs of depression, reduction of cancer, etc.), nondescript or even psychosomatic (e.g. simply, “I feel better,” as indicated subjectively by the user of the face plunge appliance), or any biochemical or cellular changes caused by the face plunge device and methods that may be associated with one or more health benefits in a human (e.g. changes in cytokine levels, production of antioxidants, flushing out of toxins from skin connective tissue, reduction in blood pressure, heart rate, pulse rate, etc.). Thus, the face plunge appliance and methods of use disclosed herein may promote one or more health benefits in a human, either macroscopically observable, subjectively “felt,” or seen/measured at the cellular/biochemical level, the effects of which may eventually be macroscopically observable.

As used herein, the term “beauty benefits” or “beauty treatment” refers broadly to any positive improvement in the visual appearance of the face, the eyes, the skin, or any other portion, or entire body, of a person using the face plunge appliance. For example, a user of the ice-cold face plunge may see a brighter skin tone, less age spots, less blemishes, decreased pore size, less redness in the eyes, tighter skin, less wrinkles, etc.

As used herein, the term “water” refers to a liquid used in the ice-cold face plunge, consisting essentially of H₂O. Thus, in the scope of the present invention, the “water” in the ice-cold face plunge of the present disclosure may include other substances, dissolved or otherwise, such as medicaments, minerals, salts, vitamins, inhalants/aromatics, sanitizers, and the like. In some instances, additives to the water in the face plunge (e.g. salts) allow for freezing point depression and the ability to have liquid water in the face plunge device at temperatures less than 32° F.

Not wishing to be bound by any particular theory regarding the underlying reasons for the observed health benefits associated with the face plunge, it is believed that a cascade of neurological and biochemical processes occur, beginning with the triggering of the CNS, followed by increases in cytokine levels in the body, which cause increases in glutathione concentrations in the body. Elevated levels of glutathione are associated with everything from tumor shrinkage in cancer patients to human longevity. Glutathione is well known to prevent cell damage by free radicals, peroxides and heavy metals. Thus the face plunge device and method herein can be used as a method of increasing glutathione concentration in a person in need thereof.

The Automatic Face Plunge Appliance

The automatic version of the face plunge of the present disclosure comprises, in combination, a water reservoir, a chilling apparatus (i.e. refrigeration system) configured to chill the water in the reservoir, and a controller. In various renditions, the controller electronically controls the chilling apparatus, which in turn controls the temperature of the water in the water reservoir. The chilling apparatus and the controller may consist entirely of digital, solid-state electronics, with any level of sophistication deemed necessary, commercially viable, and/or practical, (e.g. comprising microprocessors with programmable software, an interactive, touch-screen digital display, and so forth). The level of sophistication configured in the controller may be based at least in part on consumer demands and preferences, and cost, amongst other considerations. The chilling apparatus and/or the controller may be positioned below the water reservoir, or off to a side of the water reservoir, as desired for any particular embodiment of the appliance. These components are set out in more detail below and shown in various embodiments by way of the drawing figures.

Reservoir

The face plunge appliance comprises a water reservoir (the single term “reservoir” may be used interchangeably for simplicity). The reservoir defines the space into which the user dips his or her face to encounter chilled water, e.g. ice-cold water. The reservoir may be bowl shaped (e.g. shaped similar to a round face), having an interior volume, interior surface defining the interior volume, exterior surface, a bottom, sides and open top. The interior and exterior surfaces may be separated by any thickness of solid material or a space as discussed herein. The reservoir may be entirely smooth, with no discernable sharp corners or discernable “sides.” The open top may be fitted with a removable lid or hinged lid as desired. Such a lid may add to the aesthetics of the appliance, add additional features, assist in preventing debris from entering the water, and/or help to maintain a particular temperature for the water. The materials of construction for the lid may be the same as, or different from, the materials of construction of the reservoir. The lid may be constructed of insulating material, such as Styrofoam or plastic or other material, such as to insulate the water in the reservoir when the lid is closed. The lid may be automated and hinged, such as being configured to open or close at a particular time of day or upon transmission of a particular command such as by pressing a button, or upon the sensing of motion. In other embodiments, the lid to the face plunge may be remote controlled.

In various aspects, the reservoir may be constructed of plastic, metal, glass or various masonry materials. The reservoir may be made from, for example, Fiberglas, polyethylene, polypropylene, polycarbonate, polyethylene terephthalate, aluminum, stainless steel, brass, copper, iron, melamine, ceramics, porcelain, masonry, stone, or glass. It may be extruded, molded, forged, minted, 3D-printed, sculpted or blown, as particular materials of construction, cost and desired aesthetics may dictate. The reservoir may be insulated to any degree on any portion of the exterior surface, and may be soundproofed. Sound and/or thermal insulation may be sprayed or otherwise fixed onto the exterior surface of the reservoir, and the coated exterior surface may be placed out of sight by enclosing in an appropriately sized housing. In other aspects, the reservoir and its insulation may comprise “nested bowls,” that is, a “bowl in a bowl.” An outer bowl may provide a catch receptacle and optional drain for accidental overflow of the inner bowl, and/or provide insulation to the inner bowl. The bowl in a bowl concept for the reservoir may provide opportunity for artistic lighting enhancements with glass bowls of various transparencies and colors, and the appropriately configured lights, such as LED lighting disposed between the two bowls.

In certain aspects, the reservoir comprises a two-layer structure wherein the innermost interior surface of the inner layer defines the inner shape and volume of the reservoir, the outermost exterior surface of the outer layer defines the outer shape of the reservoir, and there exists a space between the interior and exterior layers providing a space or gap where various liquid, solid or gaseous materials may reside. This gap may comprise any volume as necessary. Liquid residing in the space between the layers of the reservoir may include a refrigerant gel or other liquid. A refrigerant gel may comprise water and any combination of a cellulosic material (e.g. hydroxyethyl cellulose), a polyacrylate, or silica. In other aspects, cooled liquid is circulated into and out of this space for chilling the water in the reservoir. In other examples, a cooling coil may be hidden within the space. Cooling coils, e.g. copper tubing with recirculating refrigerant may be submersed in a refrigerant gel within the space between layers of the reservoir. In other aspects, the cooling equipment may be placed in the space without any cooling gel or other liquid. In additional examples, the gap may be filled with insulation materials, such as Styrofoam. In some regards, the two-layer structure is equivalent to the “bowl-in-a-bowl” configuration for the reservoir, except that the peripheral edges are sealed together such that the space between the bowls is watertight.

In some configurations, it is desirable to have the reservoir removable for cleaning. For a single bowl, and no secondary reservoir in the device, the bowl may not have any plumbing connections, and thus removal may be as simple as lifting the bowl from the device, (such as from a recess in the housing where the bowl is stabilized in position). In cases where a drain, and/or water inlet/outlet are present, (such as when the device is installed—plumbed into—a home/institution, or in cases where there is a secondary reservoir and fluid communication between reservoirs), the bowl in which the face is plunged (primary reservoir) may be pulled off one or more liquid connectors, made watertight with the appropriate gaskets and/or O-rings. Removal of the reservoir may be a desirable feature so that the bowl can be cleaned, for example, placed in a dishwasher. For the nested bowl in a bowl configuration, the two bowls may be attached to one another such that the entire two-bowl assembly can be lifted from the appliance all at once, and the user doesn't have to stumble with reassembly of the nested bowl arrangement.

The “bowl in a bowl” configuration (also referred to herein as the “two-layer” configuration) also provides for a space in which to incorporate an ultraviolet (uv) light source for sanitizing the water in the reservoir. Since the water in the reservoir will be in contact with the human face, and at times exposed to the air when uncovered, the water in the reservoir is likely to eventually harbor microbes. Even though chilled water is expected to be less conducive to growth of bacteria, mold, algae, and the like, than, for example, hot water, occasional sanitizing of the water may still be desired and/or necessary, particularly in an institutional setting. Also, if the face plunge device is not plumbed directly to a water source, the reservoir may be overlooked and not drained and refilled as often as necessary (such as to meet a local or federal sanitary code), and therefore a method of sanitizing the water in the reservoir is desirable. To that end, a uv light source can be disposed between the two nested bowls, wherein the uv light can penetrate the inner bowl to irradiate the water. In various aspects of this feature, the outer bowl can be configured to block uv rays, such that the uv light cannot be seen on the outside of the outer bowl. In various embodiments, the inner surface of the outer bowl is made reflective, such that all of the uv generated from the source disposed between the bowls is deflected back through the inner bowl and through the water. The wavelengths, or range of wavelengths of the uv radiation used in sanitizing the water in the reservoir is chosen to be effective in destroying a range of organisms, such as yeast, mold, algae, amoebas and bacteria. The uv sanitizing option may be connected to a timing mechanism, or may be manually triggered. Laser lighting may provide a combination of design and function, namely sanitizing and visual light effects. In other examples, sanitizing of the facial immersion water may be achieved chemically, such as by addition of salt, chlorine or bromine or other suitable sanitizer.

The reservoir may comprise any shape, such as, for example, an oval shape that may better fit the face of the person using the appliance. The reservoir may be relatively shallow, yet configured wide and long enough to accommodate the dimensions of a typical face.

The reservoir may be configured to any volume, such as from about 0.5 liters to about 10 liters in capacity. In various embodiments, the reservoir is from about 0.5 liters to about 2 liters in capacity, such as to accommodate immersion of a human face. The fill level of the water need not be to the maximum capacity of the reservoir. For example, a 2 liter reservoir may be filled with only about 1.0 to about 1.3 liters of water such that immersion of a human face into the water will not cause any appreciable overflow of water out of the reservoir.

In other aspects, the reservoir into which the human face will be dipped may be referred to herein as the “primary reservoir” in the embodiments where the face plunge further comprises a second reservoir, referred to as the “secondary reservoir.” A secondary reservoir may be used to help maintain a cold temperature of the primary reservoir water, or for faster chilling of the water in the primary reservoir, such as more quickly down from ambient temperature. In various embodiments, the primary and secondary reservoirs are connected in fluid communication with each other, and a recirculating pump may be configured on that connection to recirculate water between the two (such as by incorporating two connecting tubes with opposite flow direction). In some cases, the secondary reservoir may be “over cooled” to the point of creating ice on its interior surfaces, and the recirculation of the water over that block ice aids in quickly dropping the temperature of the water in the primary reservoir. In these aspects, the ice in the secondary reservoir may act as a “surge producer” for chilling the water in the primary reservoir, while keeping the primary reservoir aesthetically appealing. The secondary reservoir may be from about 0.5 liters to about 1 liter in volume. In some embodiments, the primary reservoir may be about 1.3 liters in volume and the secondary reservoir about 0.75 liters in volume. In some cases, about 2 liters of total volume may exist in the ice-cold face plunge appliance.

Chilling Apparatus

The ice-cold face plunge of the present disclosure may further comprise a chilling apparatus, or refrigeration system, configured to chill the water present in the water reservoir described herein above. In the embodiments comprising primary and secondary reservoirs, the chilling apparatus may be configured to chill the water contained in either one, or both, of the reservoirs. For example, the chilling apparatus may be associated with only the secondary reservoir, thus directly chilling the water only in the secondary reservoir. In this case, the water chilled in the secondary reservoir is recirculated into the primary reservoir, which acts simply as the dipping bowl for the face. In other examples, the chilling apparatus may be configured to chill a refrigerant gel surrounding the primary reservoir. A chilling apparatus may comprise a closed system further comprising a secondary reservoir in fluid communication with the space surrounding the primary reservoir, wherein a refrigerant gel or other liquid is circulated. In various embodiments, the chilling apparatus comprises at least one thermoelectric cooler. In other embodiments, any other type of refrigeration system may be used to chill the water of the ice-cold face plunge. Although thermoelectric coolers have an advantage of not requiring circulating fluid such as Freon, the present invention is not so limited to just thermoelectric coolers, and more complicated refrigeration systems are within the scope of the present invention.

Thermoelectric Cooler

The face plunge appliance may comprise at least one thermoelectric cooler (hereinafter, “TEC”). A TEC is essentially a solid state heat pump, capable of moving heat from one side of the device to the other side when powered by an electrical current. Any number, size and type of TECs may be positioned in close proximity to, or in contact with, an outer surface of the water reservoir. For example, at least one TEC may be mounted against the exterior surface of the water reservoir such that it is in intimate contact with the reservoir. One or more flexible TECs may be shaped to the contours of the exterior surface of the reservoir and adhered thereon with an adhesive. In an embodiment comprising both primary and secondary reservoirs, one or more TECs may be configured in close proximity to, or in contact with, an outer surface of the secondary reservoir and the chilled water recirculated out to the primary reservoir on command. As mentioned, the secondary reservoir may be chilled to the point of creating ice, and the recirculation of water over the ice block is a way to surge chill the water made available in the primary reservoir. In other examples, one or more TECs are arranged on the outside of a two-layer reservoir arrangement, such that the TECs can chill the refrigerant gel present in the space between the layers of the primary reservoir.

TECs for use in the ice-cold face plunge herein may be “standard size (“C” designation) or “small size” (“S” designation) as needed, such as to accommodate a particular housing size for the overall facial bath. A single stage TEC is expected to produce a maximum temperature difference of 70° C. between its hot and cold sides. A TEC for use herein may be, for example, from about 20 watts to about 70 watts. The number of TECs used in the present chilled water facial bath may depend on various factors, such as the size of the water reservoir and fill level of water therein, a preferred/acceptable rate of cooling ambient temperature water, practicalities of heat dissipation, the wattage of each TEC, the cost of the appliance, and space/aesthetic concerns.

TECs require DC voltage, (e.g. 12 volts DC), and as such, the controller described herein below may be configured to supply the requisite DC voltage to the one or more TECs in the facial bath device to achieve and maintain a particular water temperature. The heat side of each TEC can be fitted with a heat sink to conduct heat off the hot side of the TEC. One or more fans may also be incorporated in the ice-cold face plunge as an alternate to heat sinks, or to accompany the heat sinks on the TECs. In such cases, the fan may assist in removing heat out from the facial bath and into the ambient space, such as a room. Fans for use in the ice-cold face plunge may be AC or DC powered. DC voltage can of course be reversed in polarity, and this procedure of reversing the polarity of voltage to a TEC affords the opportunity for deicing a reservoir, if needed.

In various embodiments, at use, the chilled water in the reservoir of the face plunge appliance may be about 32° F., (O° C., or close to the freezing point of the water). Lower use temperatures are possible by adding salts to the water in the appliance and taking advantage of freezing point depression. In various embodiments, the water in a single reservoir face plunge is kept cold such that solid ice and liquid water are kept in equilibrium. That is, one or more TECs are disposed at the bottom of the single reservoir where they can create sufficient localized chilling to create at least some ice adhered on the bottom inner surface of the bowl. In this way, the water is very cold, ice-cold, for the face plunge, yet the ice is physically distanced from the face such that the face won't come in contact with the ice, such as would be the case if ice were floating in the reservoir. As mentioned above, two reservoirs can be employed, and in various embodiments, the ice formation can be limited to only the secondary reservoir. In various embodiments, ice is maintained in both primary and secondary reservoirs in dual-reservoir systems, and in those examples, TECs are disposed in close proximity to or in contact with both reservoirs.

The temperature of the reservoir water at the time of use may be from about 32° F. to about 60° F., with the water optionally held at a “standby temperature” if desired, such as to conserve electricity and reduce “ramp down” time to prior to use. In this way, the temperature of the water can be ramped down to a chosen use temperature on command, or when automatically triggered by a preset timer. A standby temperature may be, for example, about 60° F., and thus the device only needs to drop from about 60° F. to about 32° F. to prep for use. Rate of cooling depends on a number of factors, most notably the volume of water to be chilled and the wattage of the one or more TECs adjacent to the water to be chilled. For example, it is estimated that to chill 1.3 liters of water from ambient to about 32° F. with only a single 20 watt TEC would take about 3 to 4 hours. Correspondingly, it would take about 1.5 to 2 hours if two 20 watt TECs are employed. Thus, in various embodiments, it may be practical to configure the appliance to have a standby mode wherein the water is kept at an intermediate temperature between use temperature (e.g. 32° F.) and ambient temperature (e.g. 72° F.), and to employ more than one 20 watt TEC or higher wattage TECs.

In various aspects, it may be desirable (and/or necessary) to prevent the water in the reservoir from freezing. As mentioned, something as simple as added salts in the water can be used for freezing point depression. More sophisticated methods to keep the water from freezing comprise magnetic fields that disrupt the association of the water molecules, a necessary step in forming solid water, i.e. ice. Magnetic fields can be disposed through the water by arranging wire coils around the reservoir bowl and supplying current through the coils. The controller discussed below can be used to power and control the magnetic fields to prevent the reservoir water from freezing. In more cost conscious variations, permanent magnets can be installed around the reservoir bowl, even inside the bowl.

Controller

In various embodiments, the ice-cold face plunge of the present invention further comprises a controller configured to electronically control the chilling apparatus described herein above. In the simplest configuration, the controller comprises only an on/off switch and power supply, designed to supply the requisite voltage/current to the chilling apparatus when the controller is switched on. For example, the controller may comprise an on/off switch, power cord and plug for 110 volt AC input, and a AC/DC step-down transformer having 110 volt AC input and 12 volt DC output. In this way, the controller, when switched on, supplies 12 volts DC to the one or more TECs present in the chilling apparatus.

In more complex and functionally diverse embodiments, the controller is configured to do much more. For example, the controller may comprise any number of circuit boards, microprocessors, and software programming to regulate the rate of water chilling, to hold desired temperature tolerances (e.g. ±2%), to afford optional deicing capability, to optionally recirculate water between primary and secondary reservoirs when there are two reservoirs, to light and interface with various LCDs, to control the sanitizing uv light source when present, power the visible light when present for pineal gland stimulation, to power and control magnetic fields to keep reservoir water from freezing, and to execute various timing aspects (such as time of day/night for the appliance to power up), amongst providing other electronic control functions. The controller may further comprise a thermostat for sensing the temperature of the reservoir water and for electronically adjusting the temperature accordingly. In some aspects, it is desirable to maintain use temperature throughout an extended period of time during which there are one or more immersions by users. A face immersion is likely to warm the water somewhat, since the human body is generally at 98.7° F., and thus the controller can chill the warmed water back to a set use temperature to be ready for the next immersion. The controller may comprise timer circuitry to chill the water in the appliance in accordance with a schedule, such as preset or entered by the user. The controller may be configured to keep the appliance ready at all times, such as by sending sufficient voltage to the chilling apparatus to keep a block of ice continually present in a secondary reservoir, or to keep the appliance in a standby mode. The controller may further include additional temperature sensors and a feedback circuit to switch on/off one or more fans in order to move built-up heat out of the appliance. In various embodiments, the controller and/or the chilling apparatus consist entirely of solid-state electronics.

The controller may also comprise one or more keypads and one or more digital displays, such as an LCD panel, configured for user interaction with the appliance. The controller may also be Bluetooth enabled to afford remote control of the appliance from a tablet or smartphone. In further aspects, displays may comprise touch-sensitive screens for easy user interface. In this way, a user may simply key in desired start times and face bath temperatures desired at particular times of the day/night directly on a keypad, or may control the appliance from a remote location over the Internet.

Housing

The various components of the face plunge appliance described herein may be housed in a suitable housing, such as to maintain safety and aesthetics. The housing may enclose one or more components, such as enclosing only the chilling apparatus and controller but not the water reservoir. In this way the housing may be used to hide and protect electronic componentry. The housing may be made of metal or plastic, for example, and may further comprise one or more portions. The housing may be metal, or plastic, or fiberboard or any other suitable material or any combinations of materials. It may be cut and fastened sheet metal, or molded plastic, or 3-D printed composite material. Multiple housing portions may be constructed from injection molded plastic, with the pieces of the housing fitting together and secured with sonic welding, screws or other fasteners when the appliance is assembled. The housing may be used to promote certain aesthetics or branding, and may further comprise labeling, decals or emblems as desired. The housing may further comprise additional functional aspects beyond enclosing and protecting the electronics, such as providing additional compartments for storing medicaments, a towel rack, a dispenser for disposable wipes, and so forth. The housing may also include exterior lighting to light the countertops around the appliance.

Optional Pineal Gland Stimulating Lighting

In various embodiments of the present invention it is desirable to have special lighting in the face plunge device that can stimulate the pineal gland (colloquially referred to as the “third eye”). The pineal gland is a very small gland positioned in the center of the brain, which produces, inter alia, serotonin and melatonin. The pineal gland is connected to photoreceptors in the eye, which is why during periods of daylight the pineal gland is not producing melatonin but is producing serotonin, whereas in periods of darkness it is producing melatonin and not serotonin. Many studies have shown the pineal gland is stimulated with visible light, and in particular, red light and blue light. Thus, the face plunge herein can be equipped with a visible light source radiating into the reservoir such that when a person immerses the face, they will be looking into the light and stimulating their pineal gland. The light source can be positioned to project light into the reservoir bowl, in which case made waterproof with the appropriate gaskets, or positioned up against the outside surface of the inner bowl and not in contact with water, or the light source may be disposed between the two bowls in a bowl in a bowl arrangement of nested bowls and not in contact with water. The light source may emit the entire visible spectrum (about 750 nm to about 380 nm), or it may selectively emit red (about 620 to about 750 nm) and/or blue light (about 450 to about 495 nm). The source may be of any intensity, and particularly an intensity that is comfortable to stare into when immersing the face. The light source may be on all the time, triggered by timer, or triggered manually, such as by depressing a momentary contact switch, like a pushbutton. In the latter embodiment, the user would immerse the face into the face plunge device, and while the face is immersed and the eyes open, push the button to turn on the visible light source.

Optional Pedestal and Base

The ice-cold face plunge of the present invention may further comprise a pedestal or other stand configured to hold the appliance at a height convenient for use by a standing person. Thus, for example, a pedestal for supporting the appliance herein may be from about 3 feet to about 5 feet tall, and appropriately configured in dimension at the top to accommodate the footprint of the device. The bottom portion of the pedestal may be configured with an integral or separate base for stability, such that the entire appliance is less prone to tipping over. The pedestal may further optionally include a locking mechanism for latching the appliance onto the pedestal to prevent accidently falling from the pedestal. The pedestal may be made from any appropriate materials, such as wood, metal or plastics, and may include any design features.

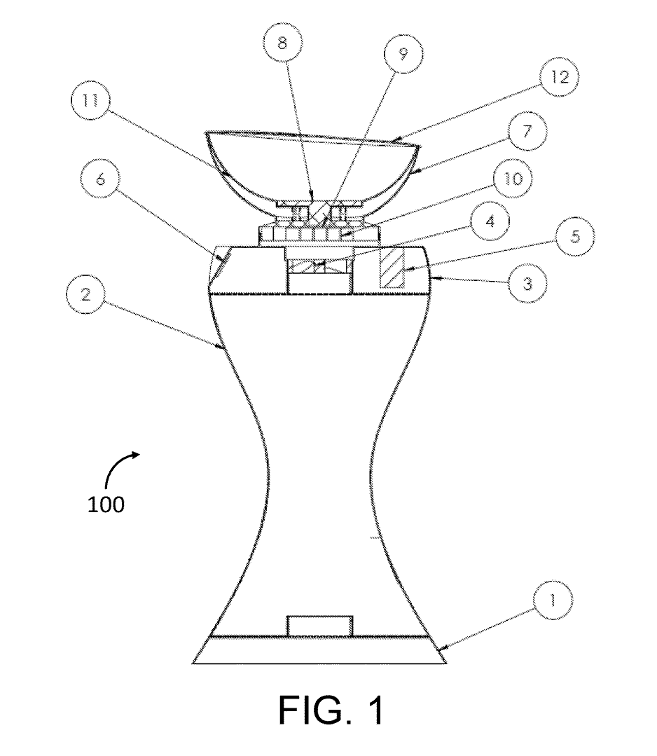

The above being noted, and with reference now to FIG. 1, an embodiment of a face plunge 100 in accordance with the present disclosure is illustrated in side view, with certain portions (e.g. housings) cut away to show various engineering details. Face plunge 100 comprises a base 1 and pedestal 2 to support the functional portion of the appliance at a convenient height. The water reservoir portion comprises the bowl in a bowl arrangement discussed above, with the inner bowl 11 being the chilled water reservoir and the outer bowl 7 adding insulation and/or decorative aspects to the appliance. Further, the space between the inner bowl 11 and outer bowl 7 may contain refrigerant gel or other liquid, insulation (foam, Fiberglas) or air. The appliance 100 further comprises a thermoelectric cooler 9, disposed in close proximity to the bottom of the inner bowl 11, such that water in the reservoir can be cooled directly. In this particular embodiment, a cold plate 11 is also employed, whereby water in the inner bowl 11 can flow through conduits within the cold plate 11. In this configuration, the thermoelectric cooler 9 can chill the water within the tubing inside the cold plate. Cold plates such as the one shown here can be made entirely of aluminum, or may be both aluminum and copper.

Directly attached to the thermoelectric cooler 9 is heatsink 10 designed to pull heat off the hot side of the thermoelectric cooler 9. The heatsink 10 is assisted by a fan 4, dimensioned and disposed such that heat can be blown from the heatsink 10 and into the ambient room around the appliance. The controller for the appliance comprises circuit board 5 and LCD 6. Not shown are the electrical connections between the various components or the electric power cord or other power supply for the controller. As configured in this embodiment, the user may interact with the appliance 100 via the LCD 6, such as to enter various settings such as timing and temperature. This particular unit further comprises a housing 3 to enclose and protect the electronics, and may further include other housing portions (not shown) to hide the chilling apparatus componentry. This embodiment is also shown to include a lid 12 that fits over the water reservoir.

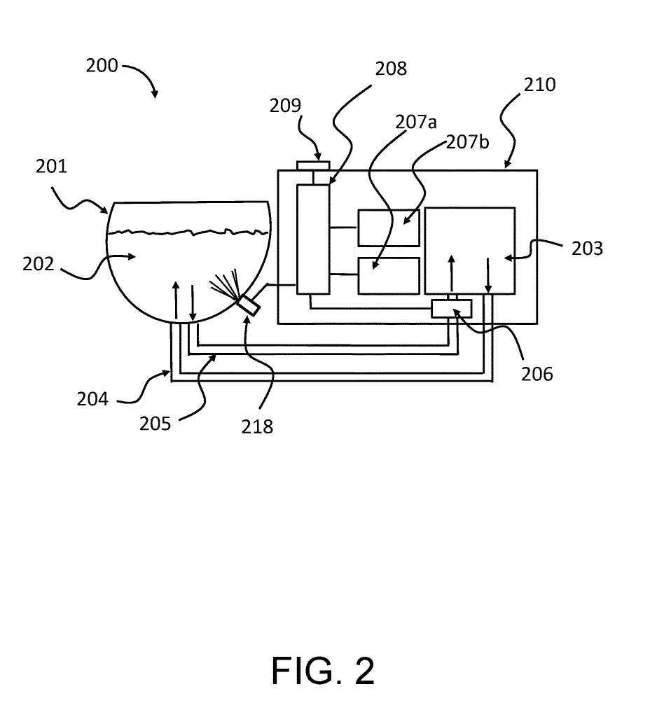

Referring now to FIG. 2, another embodiment 200 of the face plunge of the present disclosure is depicted. This particular embodiment features the two-reservoir system discussed above. As mentioned, the two-reservoir system can provide for “surge chilling” by maintaining a block of ice in the secondary (and unseen) reservoir, although in other embodiments, ice may be maintained in both primary and secondary reservoirs wherein coolers can be positioned against both reservoirs. Face plunge 200 is shown to comprise both a primary water reservoir 201 and a secondary water reservoir 203, in fluid communication with each other via the inlet/outlet pipes 204/205 and the recirculating pump 206. The water 202 in the primary water reservoir 201 is the water into which a human face is immersed in accordance with the present methods disclosed herein below. Thermoelectric coolers 207a and 207b are in close proximity to secondary reservoir 203 to chill the water therein, such as to the extent where ice is formed. Controller 208 is seen wired to the TECs (207a and 207b), to the recirculating pump 206, to the light 218 (e.g. uv source for sanitizing or visible light source for pineal gland stimulation), and to the LCD interface 209. Any number and configuration of light sources 218 may be disposed around and/or projecting into the reservoir 201 as needed for uv light and/or for visible light. The power supply is not shown, which may comprise a 110 volt AC power cord extending from controller 208 to an outlet. This particular unit is shown with a housing 210 that covers all of the componentry except for a portion of the LCD 209.

With continued reference to FIG. 2, the reservoir bowl 210 may be removable from the overall appliance, such as to clean it. It that case, O-rings and/or suitable gaskets can be employed such that one or more holes at the bottom of the bowl 201 can seal around the water inlet and outlet, forming a watertight seal.

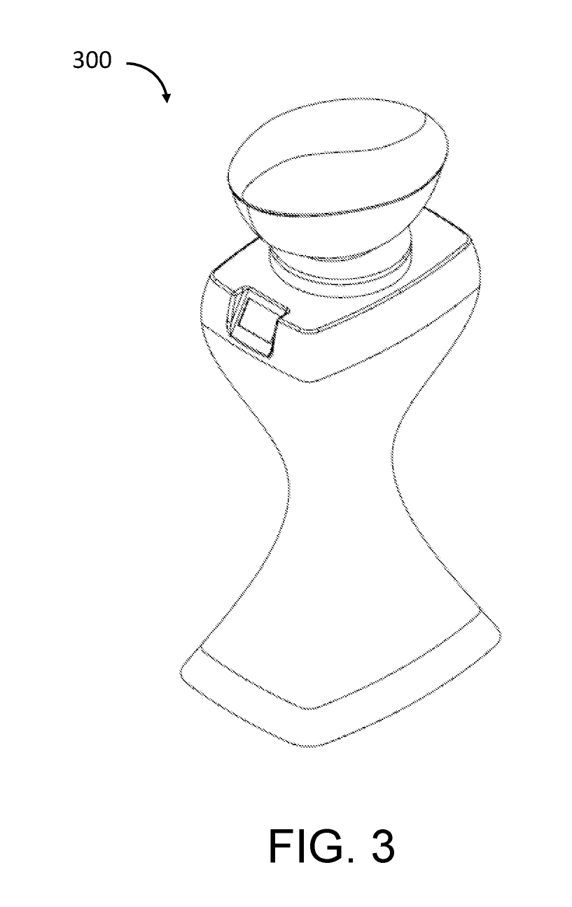

FIG. 3 depicts another embodiment 300 of the face plunge of the present disclosure.

FIG. 3 depicts another embodiment 300 of the face plunge of the present disclosure.

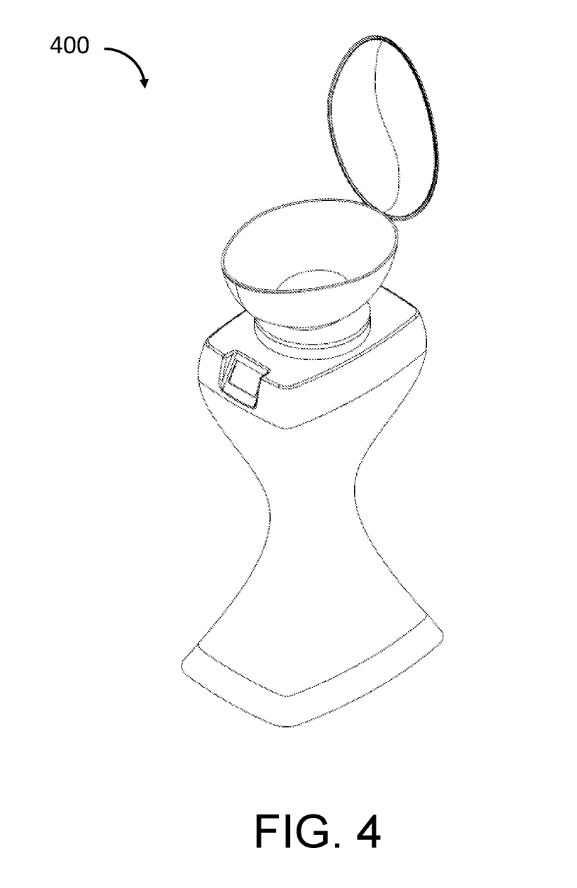

FIG. 4 depicts another embodiment 400 of the face plunge of the present disclosure.

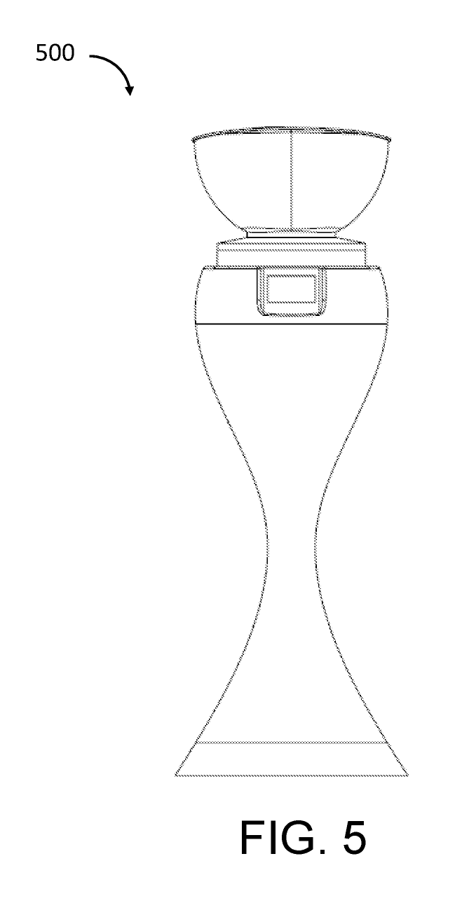

FIG. 5 depicts another embodiment 500 of the face plunge of the present disclosure.

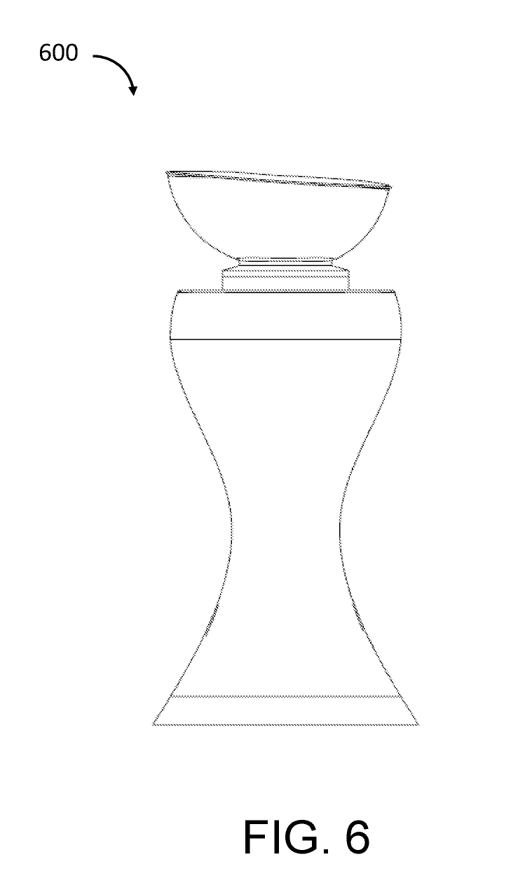

FIG. 6 depicts another embodiment 600 of the face plunge of the present disclosure.

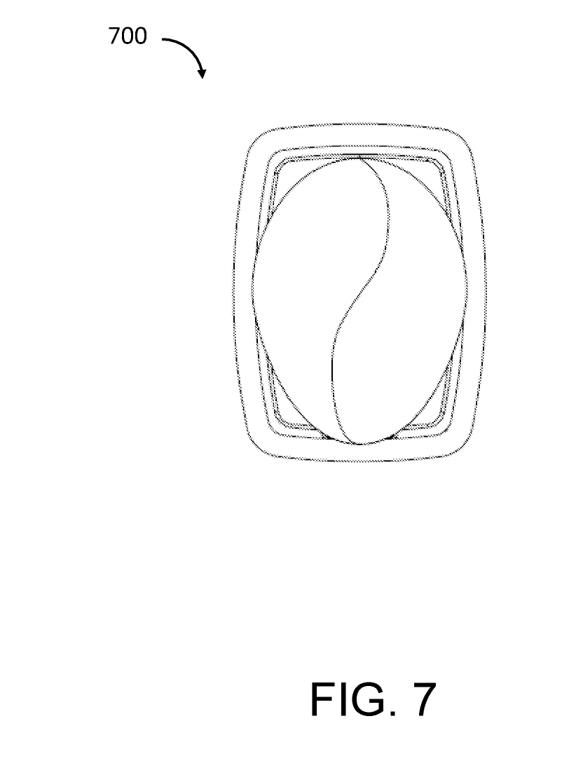

FIG. 7 depicts another embodiment 700 of the face plunge of the present disclosure.

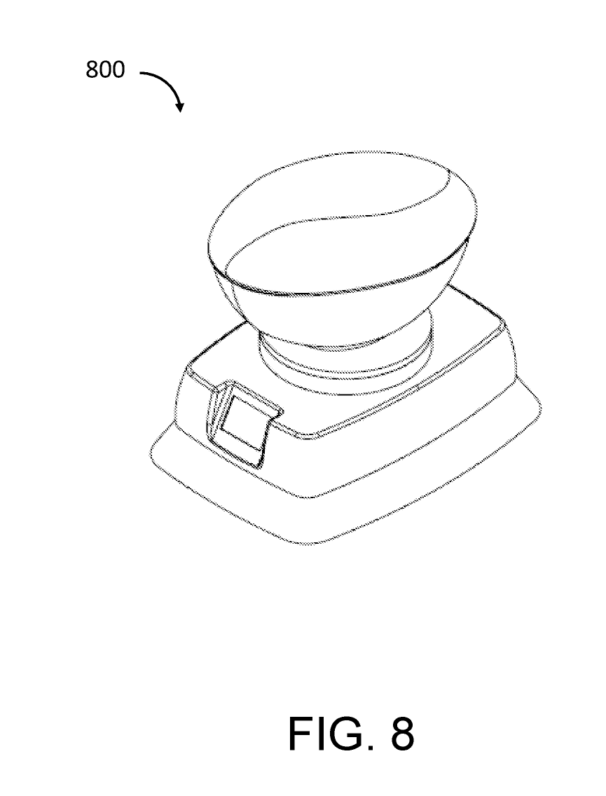

FIG. 8 depicts a tabletop embodiment 800 of the face plunge of the present disclosure.

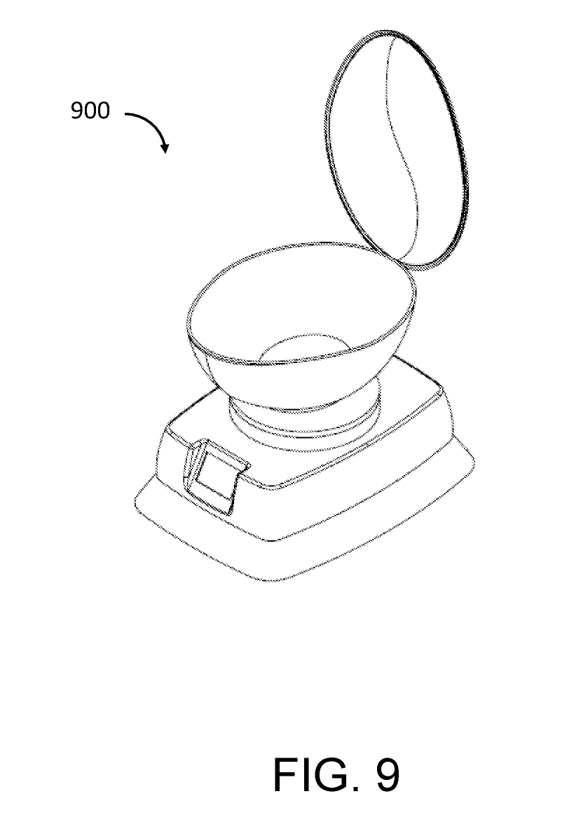

FIG. 9 depicts another tabletop embodiment 900 of the face plunge of the present disclosure.

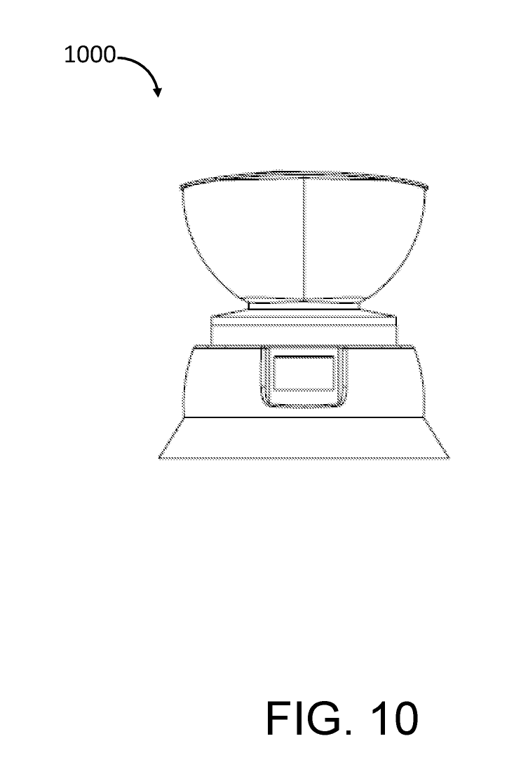

FIG. 10 depicts another tabletop embodiment 1000 of the face plunge of the present disclosure.

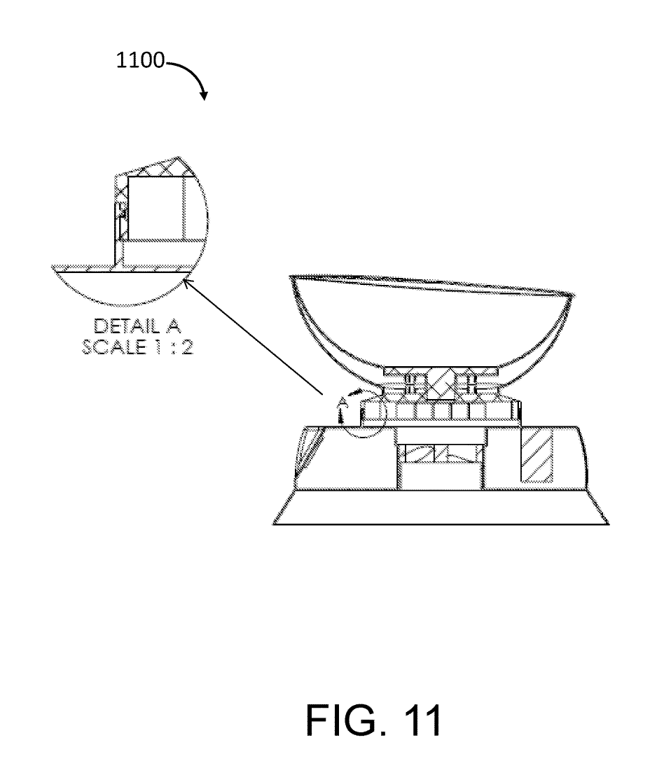

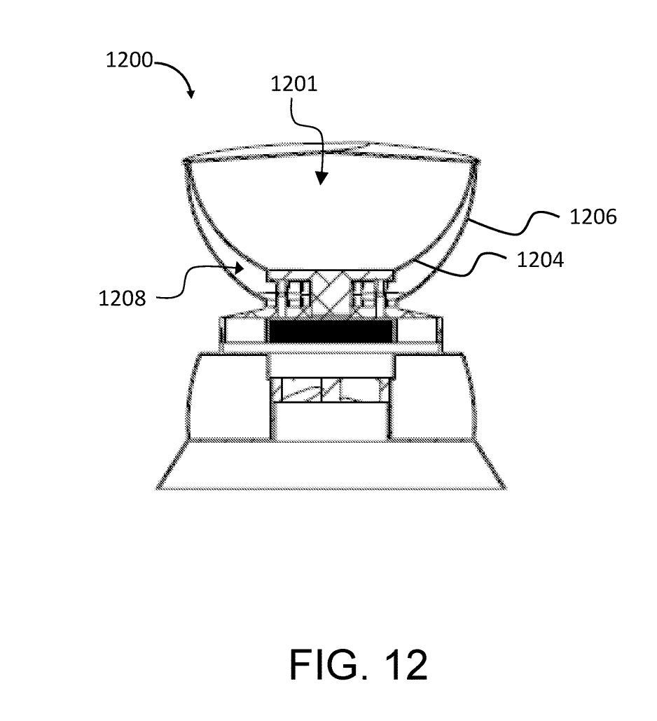

FIGS. 11 and 12 show embodiments 1100 and 1200, respectively, of the face plunge of the present disclosure, with various engineering details/componentry presented as per FIG. 1.

In various embodiments, and with continued reference to FIG. 12, the face plunge appliance 1200 of the present disclosure may comprise a primary reservoir 1201 that further comprises inner bowl 1204 and outer bowl 1206 in the previously discussed “bowl-in-a-bowl” configuration. Inner bowl 1204 may also be referred to as the inner layer or the primary reservoir, whilst the outer bowl 1206 may be referred to as the outer layer of the primary reservoir. As discussed, the space 1208 residing between inner layer 1204 and outer layer 1206 may contain a liquid, such as a refrigerant gel, insulation, or air (i.e., nothing within the gap). The refrigerant gel or other liquid may be recirculated such as to pass through a portion of the chilling apparatus, or alternatively, a chilling coil may be routed through the space 1208 to cool refrigerant in the space 1208.

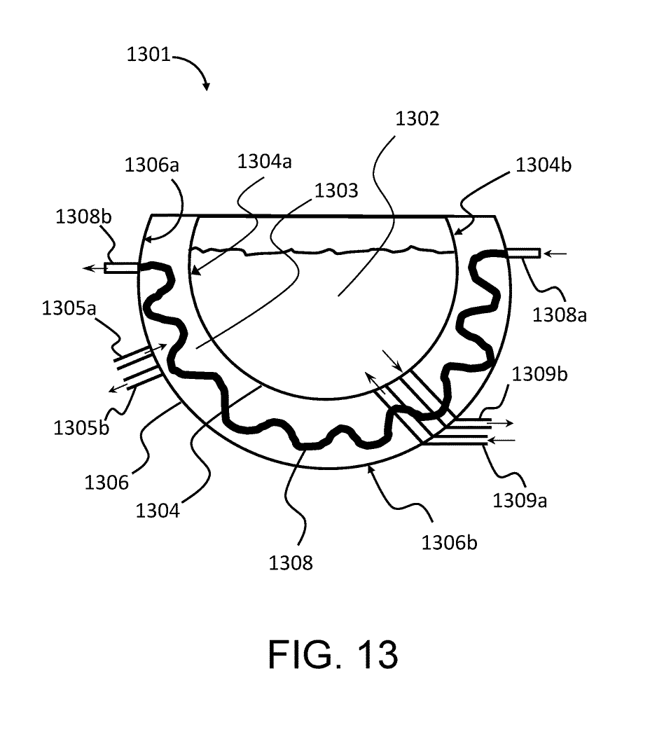

With reference now to FIG. 13, another embodiment of a primary reservoir 1301 is depicted for the face plunge appliance of the present disclosure. This embodiment illustrates various features that may be combined as necessary for any particular execution of the appliance. The primary reservoir 1301 contains a volume of water 1302 in which the user can plunge his/her face. The primary reservoir 1301 further comprises a two-layer structure, including an inner layer 1304 further comprising an interior surface 1304a and an exterior surface 1304b. As discussed, the inner layer 1304 may be of any thickness and constructed of any materials, e.g. glass. The contours of the interior surface 1304a of the inner layer 1304 defines the volume (i.e. capacity) of the primary reservoir 1301. Further, the primary reservoir 1301 comprises an outer layer 1306 further comprising an interior surface 1306a and exterior surface 1306b. The water 1302 contained in the primary reservoir 1301 may be recirculated via the inlet 1309a and outlet 1309b in fluid communication with the inner layer 1304 and thus the contents of the primary reservoir.

With continued reference to FIG. 13, the two-layer structure comprising inner layer 1304 and outer layer 1306 provides a space 1303 between the two. More specifically, there is a space 1303 between the interior surface 1306a of the outer layer 1306 and the exterior surface 1304b of the inner layer 1304. The volume of this space 1303 may be any volume desired, such as from about 100 mL to about 10 liters. As discussed, this space 1303 may be filled with a refrigerant gel or any other liquid, insulation, or simply left as an air gap. In various examples, refrigerant gel or other liquid may be recirculated into and out of the space 1303 via the inlet 1305a and outlet 1305b in fluid communication with the space 1303 and thus the material therein. In some examples, the liquid in the space 1303 is chilled water or a refrigerant gel.

Referring again to FIG. 13, the primary reservoir 1301 of the face plunge appliance may further comprise a cooling coil 1308 configured inside the space 1303 created by the two-layer or “bowl-in-a-bowl” arrangement. The cooling coil may be a length of copper tubing snaked around the inside of the space 1303. Circulating through the cooling coil may be a refrigerant, such as a liquid or condensed gas such as Freon. The recirculating of refrigerant through cooling coil 1308 is through inlet 1308a and outlet 1308b in fluid communication with the cooling coil 1308 and thus the material therein. In other examples, chilled water, cold air, liquid nitrogen, or any other material may be circulated through the cooling coil 1308 in order to chill the water 1302 residing in the primary reservoir 1301.

As discussed herein, the two-layer or bowl-in-a-bowl configuration for the primary reservoir 1301, such as depicted in FIG. 13 and several of the other drawing figures, allows for special lighting and cooling effects. For example, the inner layer 1304 may be glass, e.g. of any color, whereas the outer layer 1306 may be metal, plastic, or other non-transparent material. In this way, a light may be disposed within the space 1303 that shines through the inner layer 1304 but not out through the outer layer 1306. Such a light may be a visible wavelength light for pineal gland stimulation or a uv light for sanitization of the water 1302.

The Manual Face Plunge Device

As mentioned, a face plunge device in accordance with the present disclosure may be entirely manual, meaning devoid of any electronics such as controllers to thermostatically regulate the temperature of the facial immersion water. A manual face plunge comprises a primary reservoir having an interior surface and an exterior surface, wherein the interior surface dimensionally defines the capacity of the primary reservoir and the exterior surface defines the outer shape and overall size of the reservoir, and wherein the primary reservoir is configured in size to hold from about 0.5 liters to about 10 liters of water, and capable of at least maintaining the water at from about 32° F. to about 60° F. for a period of time sufficient to perform at least a series of facial immersions with brief pauses between immersions. In various embodiments, the primary reservoir further comprises an insulated bowl, such as a food service bowl. A manual face plunge may be as simple as a bowl or any other suitable container for water, such as a stainless steel or plastic bowl. A simple bowl can be found in many retail stores, and may be the same or similar to bowls used in food preparation and presentation. In various aspects, a manual face plunge device herein comprises a large salad bowl of sufficient dimensions to accommodate a person's face.

A manual face plunge device may comprise any type of bowl, having any shape, size and materials of construction, provided that it is large enough to accommodate facial immersion. For example, a manual face plunge device may comprise a stainless steel mixing bowl, a wooden salad bowl, or a plastic food storage bowl, such as available from Rubbermaid® or Tupperware®. Such bowls may come with a snap lid, which can be used to maintain a particular temperature of water in the bowl between facial immersions. A manual face plunge herein may further comprise decorations, such as colors and textures, or branding emblems and decals.

In various embodiments, a manual face plunge device comprises an insulated bowl. Insulated bowls are also found in the food service industry, and may be used for ice cream preparation or other cold food preparation, service or storage. For example, a manual face plunge device may comprise a stainless steel, double insulated freezer bowl for making ice cream. Other insulated bowls include double wall insulated serving bowls, such as available from American Metalcraft, Inc. In other embodiments, a manual face plunge device herein comprises a plastic Thermos®-type construction, e.g. metal outer shell protecting an internal glass inner vessel), wherein at least two layers are provided with a sealed gap there between, which may be under vacuum. Other thermal hot/cold serving bowls may be found on the Internet, and are usable as the manual facial plunge herein if large enough to accommodate the facial immersion of a human face.

In certain aspects, a manual face plunge comprises only the reservoir portion of an automatic face plunge appliance as disclosed herein. Such reservoirs include the single bowl concept, and the “bowl-in-a-bowl” concept (the two-layer reservoir). As discussed, the gap between inner and outer layers of a two-layer bowl may be filled with a refrigerant gel, solid or fibrous insulation, any liquid, a gas, vacuum, or nothing at all. In various examples, a manual face plunge device may comprise the reservoir of FIG. 13 without the various inlets and outlets or the cooling coil snaking through the gap between inner and outer layers of the reservoir.

In other variations, a manual face plunge comprises any suitable container for holding about 0.5 to about 10 liters of chilled water at from about 32° F. to about 60° F., regardless of shape. Thus, a manual face plunge may comprise a metal trough, a wooden box, a glass dish, a plastic pail, a ceramic pot, a glass casserole, a stainless steel roaster, a picnic cooler, or a sink in a bathroom. Since the manual face plunge does not include any water chilling capabilities, cold water is manually placed into the manual face plunge device, such as by transferring water from a water pitcher or by filling the manual face plunge under a faucet. A thermometer may be used to monitor the temperature of the water before, during, and after facial immersion. In some cases, it may be beneficial to add a mixture of water and ice cubes to the manual face plunge and monitor the temperature with a thermometer until the temperature equilibrates to a desired temperature between about 32° to about 60° F. Additional ice cubes may be added to the water in the manual face plunge to help maintain the desired facial immersion temperature. Or, a previously frozen freezer pack may be placed into the facial immersion water in the manual face plunge.

As per the automated appliance disclosed herein, the manual face plunge is filled with sufficient water to accommodate facial immersion, with consideration that facial immersion may cause an undesired overflow of water from the face plunge. A bathroom sink has the added advantage of a built-in overflow hole at the top, eliminating the possibility for overflow. The bathroom sink has the additional advantage of having faucets, which can be used to add a little tap water to help adjust the facial immersion water to about 32° to about 60° F.

Methods Utilizing the Manual or Automatic Face Plunge

The present disclosure provides methods for chilling the face, triggering the CNS, invigorating and energizing the person, improving general health and beauty, non-invasively treating a person therapeutically, and beauty treatment using an automatic or manual face plunge disclosed herein. All of the treatment methods disclosed herein may utilizing either a manual or automatic face plunge, e.g. anything from a stainless steel salad bowl to the most sophisticated automated appliance disclosed herein. In various embodiments, particular regimens or prescriptions are followed to chill the face to various degrees and to invigorate and refresh the user and provide the health and beauty benefits disclosed. Such regimens may be doctor or other practitioner prescribed, or may simply be empirically derived by the user of the face plunge according to personal taste and accumulated positive experiences with the appliance.

In various embodiments, the face plunge regimen comprises at least one face plunge in the AM, such as upon waking up in the morning. In other examples, a face plunge regimen comprises at least one face plunge in the PM, such as just before retiring to bed. In other examples, a face plunge regimen comprises at least one face plunge in the AM upon waking up and at least one face plunge in the PM, such as just before retiring to bed. These regimens and variations thereof can be used to reset a person's circadian rhythm.

Assays and test methods to measure the extent of the health and beauty benefits afforded by the methods using the face plunge of the present disclosure include both objective and subjective testing. Included herein are various non-limiting assays that can be used in the context of the present disclosure. It should be recognized that other testing procedures can be used, including, for example, objective and subjective procedures.

General health benefits from using the face plunge, such as “feeling better” and “having more energy,” are measured in consumer testing that includes survey data. Consumer surveys usually include a rating scale, such as a Likert scale, so that subjective feelings can be transformed into numeric values and statistically analyzed. Designing particular rating scales for consumer testing is disclosed, for example, in J. Krosnick, et al., “Designing Rating Scales for Effective Measurement in Surveys,” Chapter 6 of “Survey Measurement and Process Quality,” L. Lyberg, et al., Eds, John Wiley, New York, 1997.

Effectiveness of the methods of non-invasive therapeutic treatment include clinical methods, wherein blood samples are periodically taken from the users of the face plunge and the samples evaluated through various assay methods to measure blood levels of physiologic substances, such as cytokines.

Effectiveness of the methods of beauty treatment using the face plunge is demonstrated through specialized test methods known in the cosmetics field, as discussed herein.

Methods for Chilling the Face:

A method for chilling the face of a human comprises immersing the face in the face plunge disclosed herein for a period of time of about 1 to about 10 seconds, or for a period of time sufficient to lower the temperature of the skin of the face by at least 1° F., wherein the temperature of the water into which the face is immersed is from about 32° F. to about 60° F. prior to facial immersion.

A method for chilling the face of a human comprises immersing the face in the face plunge disclosed herein, during the day or night, for a period of time of about 1 to about 10 seconds, or for a period of time sufficient to lower the temperature of the skin of the face by at least 1° F., wherein the temperature of the water into which the face is immersed is from about 32° F. to about 60° F. prior to facial immersion, and wherein said immersion is repeated 3 to 5 times with a brief pause between immersions.

Test Methods

Reduction in skin temperature is assessed by pressing a thermometer against the skin of the user's face just prior to and just after facial immersion to obtain two temperature readings that are subtracted to obtain the difference. Alternatively, an infrared thermometer can be used to target a measurement area on the user's face, such as the center of the cheek, with a single point laser just prior to and just after facial immersion. The differences in the before and after temperature readings confirm a reduction in skin temperature of the face at greater than or equal to 1° F. upon facial immersion into about 32° F. to about 60° F. water in the facial plunge device for a period of time of about 1 to about 10 seconds.

Methods for Invigorating, Energizing a Subject and Promoting General Health:

A method of invigorating a human comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to immersion.

A method of invigorating a human during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to immersion, and wherein said immersion is repeated 3 to 5 times with a brief pause between immersions.

A method of increasing energy in a human comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to immersion.

A method of increasing energy in a human during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to immersion, and wherein said immersion is repeated 3 to 5 times with a brief pause between immersions.

A method of improving general health in a human comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion. General health improvement may further comprise a feeling of added energy for the human or a feeling of invigoration.

A method of improving general health in a human during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion, and wherein said immersion is repeated 3 to 5 times with a brief pause between immersions. General health improvement may further comprise a feeling of added energy for the human or a feeling of invigoration.

Test Methods

Surveys are provided to users of the face plunge appliance to periodically rate their energy levels, general feeling of invigoration and feeling of an improved general health (e.g. “feeling better”). The surveys include standard Likert-type 5-point rating scales where, for example, “0” indicates no change felt at all and “5” indicates a huge and substantial feeling of increased energy, invigoration or generally better health.

Methods of Non-Invasive Therapeutic Treatment:

A method of non-invasive therapeutic treatment in a human comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion. In various embodiments, the non-invasive therapeutic treatment comprises triggering the central nervous system, resetting circadian rhythm, increasing anti-inflammatory cytokine production and/or levels, decreasing inflammatory cytokine production and/or levels, increasing glutathione production and/or levels, increasing antioxidant production and/or levels, flushing out of toxins from skin connective tissue, inhibiting melanogenesis, increasing synthesis of collagen, reducing frequency/duration of colds, reducing pain, reducing swelling, reducing sinus congestion, reducing frequency/severity of acne, reducing eye redness, reducing eye irritation, reducing psoriasis, improving sleep, reducing sleep apnea, reducing severity and duration of symptoms of depression, reducing cancer, stimulating the pineal gland, reducing blood pressure levels, reducing heart rate, and reducing pulse rate.

A method of non-invasive therapeutic treatment in a human during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion, and wherein said immersion is repeated 3 to 5 times with a brief pause between immersions. In various embodiments, the non-invasive therapeutic treatment comprises triggering the central nervous system, resetting circadian rhythm, increasing anti-inflammatory cytokine production and/or levels, decreasing inflammatory cytokine production and/or levels, increasing glutathione production and/or levels, increasing antioxidant production and/or levels, flushing out of toxins from skin connective tissue, inhibiting melanogenesis, increasing synthesis of collagen, reducing frequency/duration of colds, reducing pain, reducing swelling, reducing sinus congestion, reducing frequency/severity of acne, reducing eye redness, reducing eye irritation, reducing psoriasis, improving sleep, reducing sleep apnea, reducing severity and duration of symptoms of depression, reducing cancer, stimulating the pineal gland, reducing blood pressure levels, reducing heart rate, and reducing pulse rate.

A method of triggering the central nervous system in a human comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to immersion.

A method of triggering the central nervous system in a human during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to immersion, and wherein said immersion is repeated 3 to 5 times with a brief pause between immersions.

A method of reducing the duration and severity of symptoms of depression in a human comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to immersion. Symptoms of depression include anxiety, apathy, general discontent, guilt, hopelessness, loss of interest, loss of interest or pleasure in activities, mood swings, sadness, early awakening, excessive sleepiness, insomnia, restless sleep, excessive hunger, fatigue, loss of appetite, restlessness, agitation, excessive crying, irritability, social isolation, lack of concentration, slowness in activity, thoughts of suicide, weight gain, weight loss, poor appetite, and repeatedly going over thoughts.

A method of reducing the duration and severity of symptoms of depression in a human during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to immersion, and wherein said immersion is repeated 3 to 5 times with a brief pause between immersions. Symptoms of depression include anxiety, apathy, general discontent, guilt, hopelessness, loss of interest, loss of interest or pleasure in activities, mood swings, sadness, early awakening, excessive sleepiness, insomnia, restless sleep, excessive hunger, fatigue, loss of appetite, restlessness, agitation, excessive crying, irritability, social isolation, lack of concentration, slowness in activity, thoughts of suicide, weight gain, weight loss, poor appetite, and repeatedly going over thoughts.

A method of increasing antioxidant levels, such as glutathione, in a human comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion.

A method of increasing antioxidant levels, such as glutathione, in a human during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion, and wherein said immersion is repeated 3 to 5 times with a brief pause between immersions.

A method of increasing anti-inflammatory cytokine levels in a human comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to immersion. In various embodiments, the anti-inflammatory cytokines comprise at least one of IL-1 receptor antagonist, IL-4, IL-10, IL-11, IL-13 and TGF-β.

A method of increasing anti-inflammatory cytokine levels in a human during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion, and wherein said immersion is repeated 3 to 5 times with a brief pause between immersions. In various embodiments, the anti-inflammatory cytokines comprise at least one of IL-1 receptor antagonist, IL-4, IL-10, IL-11, IL-13 and TGF-β.

A method of decreasing inflammatory cytokine levels in a human comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to immersion. In various embodiments, the inflammatory cytokines comprise at least one of IL-1, TNF-α, and IFN-γ.

A method of decreasing inflammatory cytokine levels in a human during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion, and wherein said immersion is repeated 3 to 5 times with a brief pause between immersions. In various embodiments, the inflammatory cytokines comprise at least one of IL-1, TNF-α, and IFN-γ.

A method of stimulating the pineal gland in a human comprises immersing the face of the human in need thereof with eyes open in the face plunge disclosed herein equipped with at least one visible light source in the ON position, for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion.

A method of stimulating the pineal gland in a human during the daytime comprises immersing the face of the human in need thereof with eyes open in the face plunge disclosed herein equipped with at least one visible light source in the ON position, for a period of time of about 1 to about 10 seconds, or for a period of time to stimulate serotonin production in the pineal gland, or for a period of time sufficient to lower the temperature of the skin of the face by at least 1° F., wherein the temperature of the water into which the face is immersed is at about 32° F. to about 60° F. prior to facial immersion, and wherein said immersion and eye exposure to the visible light is repeated 3 to 5 times with a brief pause between immersions.

Circadian rhythm refers to the approximately 24 cycle in the physiological processes of humans and many other life forms. Circadian rhythms are important in determining sleep and feeding patterns. Although circadian rhythms are endogenous to the person, the rhythms are adjusted to local environments by cues such as light and temperature. Travel across time zones or across regions of sunlight and darkness in a single day, amongst other activities including stress and alcohol, can affect a person's circadian rhythm. For example, travelers may experience fatigue, disorientation and insomnia. Use of the face plunge of the present disclosure resets the circadian rhythm of the user, and thus is useful for frequent travelers or others who experience the symptoms from disruption to their natural circadian rhythms. In various examples, the person in need thereof uses the face plunge appliance of the present disclosure either AM or PM, or both, for as many days as necessary to reset their circadian rhythm and thus their normal wake and sleep cycle.

A method of resetting circadian rhythm in a human during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to immersion.

A method of resetting circadian rhythm in a human during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion, and wherein said immersion is repeated 3 to 5 times with a brief pause between immersions.

In various embodiments, the method comprises immersing the face at least once in the AM, such as upon waking up in the morning. For travelers that are trying to reset their circadian rhythm to the local time zone, the traveler uses the face plunge device in the AM local time, such as at the first sign of daylight in the local time zone. The traveler may use the face plunge at first light even if not having slept during the night. In other embodiments, the method of resetting circadian rhythm comprises immersing the face at least once in the PM, such as when preparing to retire to bed at night. For travelers that are trying to reset their circadian rhythm to the local time zone, the traveler uses the face plunge device in the PM local time, such as when most are going to bed in the local time zone. The traveler may use the face plunge upon preparing to retire in the PM local time even if sleeping on and off during the day. In other embodiments, the method of resetting circadian rhythm comprises using the face plunge both in the AM and in the PM.

A method of reducing eye redness and/or eye irritation in a human comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F.

A method of reducing eye redness and/or eye irritation in a human during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F., and wherein said immersion is repeated 3 to 5 times with a brief pause between immersions.

Test Methods

For surveying users of the face plunge for effectiveness in reducing the duration and severity of symptoms of depression, the surveys include a self-rating scale, such as the Beck Depression Inventory (BDI), which is used to measure the severity of depressive symptoms that the test taker is experiencing “at that moment.” Face plunge users are instructed to self-rate at a prescribed time period, such as daily. Surveys for consumers using the device to reset circadian rhythm or to stimulate the pineal gland include Likert-type rating scales to rate sleep patterns since the pineal gland produces melatonin known to modulate sleep patterns in both circadian and seasonal cycles. Surveys for users of the device to reduce eye redness and/or eye irritation also include Likert-type rating scales for the user to self-rate what they see in the mirror and feel regarding their eyes. For example, users can rank their eye redness/irritation each day on a scale of 0 (no redness/irritation) to 5 (severe redness/irritation) during the consumer study of the face plunge.

Assay Methods

Participants using the face plunge as prescribed in the methods above are subjected to a periodic blood draw, and the blood cytokine levels determined by assaying the samples in a clinical laboratory test. Exemplary methods to determine blood cytokine levels are found in T. Takahashi, et al., Rinsho Byori (The Japanese Journal of Clinical Pathology), 55(3), 272-9, 2007.

Methods of Beauty Treatment:

A method of beauty treatment in a human comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion. In various embodiments, methods of beauty treatment comprise reducing skin redness, moisturizing and hydrating skin, improving skin clarity, reducing freckles, reducing age spots, reducing skin dryness, reducing fine lines, reducing pore size, improving skin smoothness, improving skin tone, reducing wrinkles, increasing skin firmness, increasing skin softness, stimulating production of new skin cells, and improving skin suppleness.

A method of beauty treatment in a human during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion, and wherein said immersion is repeated 3 to 5 times with a brief pause between immersions. In various embodiments, methods of beauty treatment comprise reducing skin redness, moisturizing and hydrating skin, improving skin clarity, reducing freckles, reducing age spots, reducing skin dryness, reducing fine lines, reducing pore size, improving skin smoothness, improving skin tone, reducing wrinkles, increasing skin firmness, increasing skin softness, stimulating production of new skin cells, and improving skin suppleness.

A method of reducing pore size on the skin of a human face comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion.

A method of reducing pore size on the skin of a human face during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion, and wherein said immersion is repeated 3 to 5 times with a brief pause between immersions.

A method of improving skin tone of a human face comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion.

A method of improving skin tone of a human face during the day or night comprises immersing the face of the human in need thereof in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion, and wherein said immersion is repeated 3 to 5 times with a brief pause between immersions.

A finishing protocol for a person at the end of a spa treatment comprises immersing the face of the person in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion.

A finishing protocol for a facial spa treatment comprises immersing the face of the person in the face plunge disclosed herein for a period of time of about 1 to about 10 seconds, or for a period of time sufficient to lower the temperature of the skin of the face by at least 1° F., wherein the temperature of the water into which the face is immersed is at about 32° F. to about 60° F. prior to facial immersion.

Assays and Test Methods

An assay to measure the reduction of skin redness comprises using a chromameter, (e.g. a CR-400 or CR-410 Chroma Meter from Konica Minolta), which is capable of reading color differences. These instruments are portable and can be issued to consumers using the face plunge at home. Redness can be assessed using the a* values of the meter. The a* value measures changes in skin color in the red region. Repeat measurements are taken at regular intervals to determine the face plunge's ability to reduce redness, inflammation, or skin irritation.

Skin moisture/hydration benefits from using the face plunge can be measured by using impedance measurements with the dermal phase meter, such as from NOVA. The impedance meter measures changes in skin moisture content. The outer layer of the skin has distinct electrical properties, and when skin is dry it conducts electricity very poorly. Conversely, as skin becomes more hydrated the conductivity increases. Consequently, changes in skin impedance can be used to assess changes in skin hydration.

Skin clarity and the reduction in freckles and age spots can be evaluated using a chromameter, such as the CR-400 or CR-410 Chroma Meter from Konica Minolta. Changes in skin color can be assessed using the a* values of the Minolta Chroma Meter. The a* value measures changes in skin color in the red region. The measurements can be made on each side of the face and averaged, as left and right facial values. Skin clarity can also be measured using the Minolta Meter. The measurement is a combination of the a*, b, and L values from the Minolta Meter relating to skin brightness and correlating well with skin smoothness and hydration.

Skin dryness, surface fine lines, skin smoothness, and skin tone can be evaluated with clinical grading techniques. For example, clinical grading of skin dryness can be determined by a five point standard Kligman Grading Scale: (0) skin is soft and moist; (1) skin appears normal with no visible dryness; (2) skin feels slightly dry to the touch with no visible flaking; (3) skin feels dry, tough, and has a whitish appearance with some scaling; and (4) skin feels very dry, rough, and has a whitish appearance with scaling. Evaluations are made independently by two clinicians and averaged, or alternatively, the grading scale can be incorporated into surveys taken periodically by the users of the face plunge appliance.

Clinical grading of skin tone can be performed via a ten point analog numerical scale: (10) even skin of uniform, pinkish brown color. No dark or scaly patches upon examination with a hand held magnifying lens. Fine texture of the skin very uniform upon touch; (7) even skin tone observed without magnification. No scaly areas, but slight discolorations either due to pigmentation or erythema. No discolorations more than 1 cm in diameter; (4) both skin discoloration and uneven texture easily noticeable. Slight scaling. Skin rough to the touch in some areas; and (1) uneven skin coloration and texture. Numerous areas of scaliness and discoloration, either hypo-pigmented, erythremic or dark spots. Large areas of uneven color more than 1 cm in diameter. Evaluations are made independently by two clinicians and averaged, or alternatively, the 10-point scale is incorporated into surveys taken periodically by the users of the face plunge appliance.

Clinical grading of skin smoothness can be analyzed via a 10-point analog numerical scale: (10) smooth, skin is moist and glistening, no resistance upon dragging finger across surface; (7) somewhat smooth, slight resistance; (4) rough, visibly altered, friction upon rubbing; and (1) rough, flaky, uneven surface. Evaluations are made independently by two clinicians and averaged, or alternatively, the 10-point scale is incorporated into surveys taken periodically by the users of the face plunge appliance.

Skin firmness can be measured using ballistometry, such as with a Hargens ballistometer that evaluates the elasticity and firmness of the skin by dropping a small body onto the skin and recording its first two rebound peaks. In ballistometry, a small lightweight probe with a relatively blunt tip (e.g. 4 mm² contact area) is used. The probe penetrates slightly into the skin and results in measurements that are dependent upon the properties of the outer layers of the skin, including the stratum corneum and outer epidermis and some of the dermal layers. The subject may be placed in a supine position such that this test procedure can be easily performed on the face. In various aspects, users of the face plunge appliance may periodically visit an office where the measurements can be taken.

Skin softness/suppleness can be evaluated using the Gas Bearing Electrodynamometer, an instrument that measures the stress/strain properties of the skin. The viscoelastic properties of skin correlate with skin moisturization. Measurements can be obtained on the predetermined site on the cheek area by attaching the probe to the skin surface with double-sided tape. A force of approximately 3.5 gm can be applied parallel to the skin surface and the skin displacement is accurately measured. Skin suppleness can then be calculated and is expressed as DSR (Dynamic Spring Rate in gm/mm).

It will be apparent to those skilled in the art that various modifications and variations are possible without departing from the spirit or scope of the disclosure. Thus, it is intended that the present disclosure cover the modifications and variations of this disclosure provided they come within the scope of the appended claims and their equivalents.

Likewise, numerous characteristics and advantages have been set forth in the preceding description, including various alternatives together with details of the structure and function of the appliance and/or methods. The disclosure is intended as illustrative only and as such is not intended to be exhaustive. It will be evident to those skilled in the art that various modifications may be made, especially in matters of structure, materials, elements, components, shape, size and arrangement of parts including combinations within the principles of the disclosure, to the full extent indicated by the broad, general meaning of the terms in which the appended claims are expressed. To the extent that these various modifications do not depart from the spirit and scope of the appended claims, they are intended to be encompassed therein.

1. A face plunge device for chilling the face of a human, said device comprising:
a primary reservoir having an interior surface and an exterior surface; and
a volume of water in said primary reservoir, said water at a temperature of from about 32° F. to about 60° F.,
wherein immersion of the face in the water for a period of time of from about 1 second to about 10 seconds lowers the temperature of the skin of the face by at least 1° F.

2. The face plunge of claim 1, further comprising a chilling apparatus configured to chill the water and a controller configured to electronically control the chilling apparatus.

3. The face plunge device of claim 1, wherein said volume of water is from about 0.5 liters to about 10 liters.

4. The face plunge device of claim 1, wherein said primary reservoir comprises a two-layer structure of inner and outer layers, wherein an innermost interior surface of the inner layer defines a volume of the primary reservoir, the outermost exterior surface of the outer layer defines an outer shape of the primary reservoir, and a space between the interior and exterior layers.

5. The face plunge device of claim 2, wherein said chilling apparatus comprises at least one thermoelectric cooler (TEC) disposed in close proximity to, or in contact with, the exterior surface of the primary reservoir.

6. The face plunge device of claim 2, further comprising a secondary reservoir in fluid communication with said primary reservoir.

7. The face plunge device of claim 2, wherein said chilling apparatus is configured to chill water present in the secondary reservoir and to circulate the water between the primary and secondary reservoirs via a pump.

8. The face plunge device of claim 7, wherein said chilling apparatus comprises at least one thermoelectric cooler (TEC) disposed in close proximity to, or in contact with, the secondary reservoir.

9. The face plunge device of claim 2, further comprising a visible light source configured and disposed to project visible light into the primary reservoir and into the eyes of the human during facial immersion, said visible light having wavelengths in the range of from about 450 to about 495 nm or from about 620 to about 750 nm.

10. The face plunge device of claim 2, wherein said controller thermostatically maintains said water temperature of from about 32° F. to about 60° F.

11. A method of non-invasive therapeutic treatment in a human, said method comprising immersing the face of the human in need thereof in the face plunge device of claim 1 for a period of time of from about 1 to about 10 seconds, or for a period of time sufficient to lower the temperature of the skin of the face by at least 1° F., wherein the temperature of the water into which the face is immersed is at about 32° F. to about 60° F. prior to immersion.

12. The method of claim 11, wherein said immersion is repeated 3 to 5 times with a brief pause between immersions.

13. The method of claim 11, wherein the non-invasive therapeutic treatment comprises at least one of triggering the central nervous system, resetting circadian rhythm, increasing anti-inflammatory cytokine production and/or levels, decreasing inflammatory cytokine production and/or levels, increasing glutathione production and/or levels, increasing antioxidant production and/or levels, flushing out of toxins from skin connective tissue, inhibiting melanogenesis, increasing synthesis of collagen, reducing frequency/duration of colds, reducing pain, reducing swelling, reducing sinus congestion, reducing frequency/severity of acne, reducing eye redness, reducing eye irritation, reducing psoriasis, improving sleep, reducing sleep apnea, reducing severity and duration of symptoms of depression, reducing cancer, reducing blood pressure levels, reducing heart rate, and reducing pulse rate.

14. The method of claim 11, wherein said symptoms of depression comprise at least one of anxiety, apathy, general discontent, guilt, hopelessness, loss of interest, loss of interest or pleasure in activities, mood swings, sadness, early awakening, excessive sleepiness, insomnia, restless sleep, excessive hunger, fatigue, loss of appetite, restlessness, agitation, excessive crying, irritability, social isolation, lack of concentration, slowness in activity, thoughts of suicide, weight gain, weight loss, poor appetite, and repeatedly going over thoughts.

15. The method of claim 11, wherein said anti-inflammatory cytokines comprise at least one of IL-1 receptor antagonist, IL-4, IL-10, IL-11, IL-13 and TGF-β.

16. The method of claim 11, wherein said inflammatory cytokines comprise at least one of IL-1, TNF-α, and IFN-γ.

17. A method of beauty treatment in a human, said method comprising immersing the face of the human in need thereof in the face plunge device of claim 1 for a period of time of from about 1 to about 10 seconds, or for a period of time sufficient to lower the temperature of the skin of the face by at least 1° F., wherein the temperature of the water into which the face is immersed is at about 32° F. to about 60° F. prior to immersion.

18. The method of claim 17, wherein said immersion is repeated 3 to 5 times with a brief pause between immersions.

19. The method of claim 17, wherein said beauty treatment comprises at least one of reducing skin redness, moisturizing and hydrating skin, improving skin clarity, reducing freckles, reducing age spots, reducing skin dryness, reducing fine lines, reducing pore size, improving skin smoothness, improving skin tone, reducing wrinkles, increasing skin firmness, increasing skin softness, stimulating production of new skin cells, and improving skin suppleness.

20. A method of invigorating or increasing energy in a human, said method comprising immersing the face of the human in need thereof in the face plunge device of claim 1 for a period of time of from about 1 to about 10 seconds, or for a period of time sufficient to lower the temperature of the skin of the face by at least 1° F., wherein the temperature of the water into which the face is immersed is at about 32° F. to about 60° F. prior to immersion.

21. The method of claim 20, wherein said immersion is repeated 3 to 5 times with a brief pause between immersions.

22. A method of resetting circadian rhythm in a human during the day or night, said method comprising immersing the face of the human in need thereof in the face plunge device of claim 1 for a period of time of from about 1 to about 10 seconds, or for a period of time sufficient to lower the temperature of the skin of the face by at least 1° F., wherein the temperature of the water into which the face is immersed is at about 32° F. to about 60° F. prior to immersion.

23. The method of claim 22, wherein said immersion is repeated 3 to 5 times with a brief pause between immersions.

24. The method of claim 22, wherein said immersion is conducted in the AM local time of the human in need thereof, for as many days necessary to restore normal sleep and wake cycles.

25. The method of claim 22, wherein said immersion is conducted in the PM local time of the human in need thereof, for as many days necessary to restore normal sleep and wake cycles.

26. The method of claim 22, wherein the human in need thereof plunges his/her face in the AM and in the PM for at least as many days necessary to restore normal sleep and wake cycles.

27. A method of triggering the CNS of a person, invigorating a person, increasing energy in a person, achieving instant alertness in a person, mending the circadian rhythm in a person, providing non-invasive therapeutic treatment to a person, or providing beauty treatment to a person, said method comprising immersing the face of the person in need thereof in a volume of chilled water for a period of time of from about 1 to about 10 seconds, or for a period of time sufficient to lower the temperature of the skin of the face by at least 1° F., wherein the temperature of the chilled water into which the face is immersed is at about 32° F. to about 60° F. prior to immersion.

28. The method of claim 27, wherein said immersion is repeated 3 to 5 times with a brief pause between immersions.

29. A method of triggering the CNS of a person, invigorating a person, increasing energy in a person, achieving instant alertness in a person, mending the circadian rhythm in a person, providing non-invasive therapeutic treatment to a person, or providing beauty treatment to a person, said method comprising:
immersing the face of the person in need thereof in a volume of chilled water contained in a face plunge device for a period of time of from about 1 to about 10 seconds, or for a period of time sufficient to lower the temperature of the skin of the face by at least 1° F.,
wherein said face plunge device comprises: a primary reservoir having an interior surface and an exterior surface; a chilling apparatus configured to chill the volume of water; and a controller configured to electronically control the chilling apparatus,
wherein said volume of chilled water is contained within said primary reservoir, and
wherein the temperature of the chilled water into which the face is immersed is at from about 32° F. to about 60° F. prior to facial immersion.

30. The method of claim 29, wherein said immersion is repeated 3 to 5 times with a brief pause between immersions.
